(19) United States
(12) Patent Application Publication
(10) Pub. No.: US 2017/0354793 A1
(43) Pub. Date: Dec. 14, 2017

(54) RESPIRATORY DEVICE TO TRACHEOSTOMY TUBE CONNECTOR

(71) Applicant: Alaaeldin Soliman, Holmdel, NJ (US)

(72) Inventor: Alaaeldin Soliman, Holmdel, NJ (US)

(21) Appl. No.: 15/177,434

(22) Filed: Jun. 9, 2016

(51) Int. Cl. A61M 16/08; A61M 16/04; A61M 16/20; A61M 16/00

(52) U.S. Cl. CPC A61M 16/0816; A61M 16/0051; A61M 16/0465; A61M 16/0057; A61M 16/208; A61M 2205/3334; A61M 2016/003; A61M 2205/35

(57) ABSTRACT

A respiratory device (e.g., BiPAP machine) to tracheostomy tube connector includes a first component including a first hollow channel through which air is pumped from a respiratory device in one direction, and a second component connected to the first component, wherein the second component includes a second hollow channel through which air moves in two directions to and from a tracheostomy tube, and an opening through which air is vented upon return from the tracheostomy tube. The connector further includes a valve positioned in between the first component and the second component; and a sensor seated in the second component and adjacent to the opening, wherein the sensor detects any of a flow of air in the second component and a motion of the valve.

### BACKGROUND

### Technical Field

The embodiments herein generally relate to medical devices, and more particularly to respiratory mechanical ventilation devices.

### Description of the Related Art

Bilevel positive airway pressure (BiPAP) is a mechanical ventilation device that assists patients in breathing by delivering measured amounts of pressure into their lungs to keep the airways open and the lung's tissue inflated for a better exchange of oxygen and carbon dioxide. Generally, there are two types of machines that are used to help ventilating a patient suffering from mild chronic respiratory failure: (i) a non-invasive BiPAP machine that delivers its help via a tight mask held against the patient's nose/mouth, and which could be used at home during the times of need, and (ii) an invasive, complex, and costly ventilator machine that delivers its help via a tube inserted into the patient's trachea (windpipe) through a hole made in the neck named tracheostomy, and which is typically provided only in health care facilities, not because of the tracheostomy care but rather because of the ventilator machine care. The essential concept of mechanical ventilation is to establish a closed circuit of pressure composed of the ventilator machine, connecting tube—or tight mask—and the patient's lungs.

In BiPAP machines, the pressure changes during the respiratory cycle (inhalation-exhalation). For this reason, among others, “BiPAP to tracheostomy tube” is not currently approved by the U.S. Food & Drug Administration (FDA) since there are no alarms on the devices to warn of a pressure change or a disconnection of the various tubes/seals. Often, patients with a tracheostomy must sign a medical waiver indicating that they understand this aspect of the BiPAP devices being connected to a tracheostomy tube without an alarm. Another problem with BiPAP machines connected to a tracheostomy tube is the lack of a mechanism that allows the volume of air inhaled in the patient's lungs during inhalation to exit during exhalation, so the air will not be trapped in the lungs. Ventilators can accomplish this. However, ventilators tend to be large, rather noisy and more medically complex for patients than a simpler BiPAP machine. Accordingly, there is a need for a device that allows a large population of patients with tracheostomy tubes and mild respiratory failure who do not need the support of an advanced, large, heavy, costly and more complicated ventilator machine, but rather allow this patient population to benefit from a smaller, less complicated ventilating machine, such as a BiPA that can provide a similar ventilation support.

### SUMMARY

In view of the foregoing, an embodiment herein provides a respiratory device to tracheostomy tube connector comprising a first component comprising a first hollow channel through which air is pumped from a respiratory device in one direction; and a second component connected to the first component, wherein the second component comprises a second hollow channel through which air moves in two directions to and from a tracheostomy tube; and an opening through which air is vented upon return from the tracheostomy tube. The connector further comprises a valve positioned in between the first component and the second component; and a sensor seated in the second component and adjacent to the opening, wherein the sensor detects any of a flow of air in the second component and a motion of the valve. The respiratory device may comprise BiPAP machine.

The sensor may comprise an air flowmeter. The sensor may comprise a detector. The detector may detect air pressure in the second component. The detector may detect the motion of the valve. The sensor may comprise a wire that transmits electrical signals to and from the detector. The sensor may comprise a module. The module may provide power to the sensor. The module may electrically connect the wire to the detector. The wire may comprise an antenna. The wire may extend from the sensor away from the second component. The sensor may comprise a speaker that outputs audio. The sensor may comprise a light indicator.

The second component may comprise a tube interface portion configured to engage the tracheostomy tube; and a body portion comprising the opening and the sensor, wherein the second hollow channel extends from the tube interface portion to the body portion. An outer diametrical width of the tube interface portion may be less than an outer diametrical width of the body portion. The connector may further comprise a lip separating the tube interface portion from the body portion. The tracheostomy tube does not connect to a respiration mask. The valve may comprise a flexible central portion that articulates from an open to closed position, wherein the flexible central portion covers the opening when the valve is in the open position. When the valve is in the closed position, air returning from the tracheostomy tube may be prevented from entering into the first component.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a perspective view of a tracheostomy sensor connector according to an embodiment herein;

FIG. 2 illustrates a front view of the connector of FIG. 1 according to an embodiment herein;

FIG. 3 illustrates a right side view of the connector of FIG. 1 according to an embodiment herein;

FIG. 4 illustrates a left side view of the connector of FIG. 1 according to an embodiment herein;

FIG. 5 illustrates a top view of the connector of FIG. 1 according to an embodiment herein;

FIG. 6 illustrates a bottom view of the connector of FIG. 1 according to an embodiment herein;

FIG. 7 illustrates a cross-sectional perspective view of the connector of FIG. 1 according to an embodiment herein;

FIG. 8 illustrates a cross-sectional exploded perspective view of the connector of FIG. 7 according to an embodiment herein;

FIG. 9 illustrates an exploded perspective view of the connector of FIG. 7 according to an embodiment herein;

FIG. 10 illustrates a perspective system view of the tracheostomy sensor connector of FIG. 1 connected to a trach tube and a respiratory device tube according to an embodiment herein;

FIG. 11A illustrates a perspective view of the sensor of the connector of FIG. 1 according to an embodiment herein;

FIG. 11B illustrates a top view of the sensor of FIG. 11A according to an embodiment herein;

FIG. 11C illustrates a bottom view of the sensor of FIG. 11A according to an embodiment herein; and

FIG. 11D illustrates a cross-sectional view of the sensor cut along line A-A of FIG. 11A according to an embodiment herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

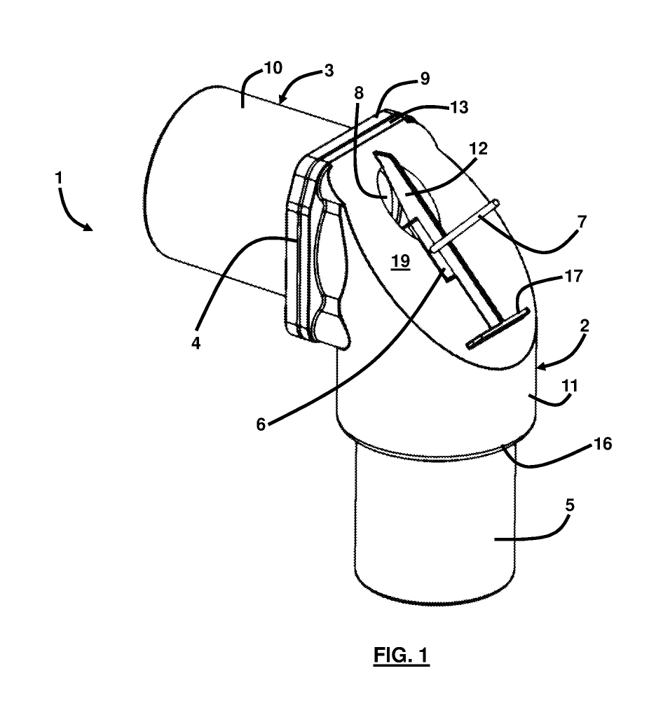

As mentioned, there is a need for a device that allows a smaller, less complicated respiratory machine, such as a BiPAP, to be used to provide the similar respiratory support of a larger, heavier, and more complicated and costly ventilator machine for tracheostomy patients. The embodiments herein provide a connector that allows a BiPAP machine to be connected to a tracheostomy tube and without requiring a respiratory mask. The embodiments herein further provide an alarm and mechanism to exit the inhaled air. Referring now to the drawings, and more particularly to FIGS. 1 through 11D, where similar reference characters denote corresponding features consistently throughout the figures, there are shown preferred embodiments.

FIG. 1 illustrates a perspective view of a tracheostomy sensor connector 1 according to an embodiment herein. The connector 1 comprises a hollow vent component 2 connected to a hollow trach component 3. A valve 4 is disposed between the vent component 2 and the trach component 3. The vent component 2 comprises a vent body 11 and a tube interface 5, with a neck 16 separating the vent body 11 from the tube interface 5. A sensor 6 is positioned in the vent component 2 and is connected to a wire 7. The wire 7 may be configured as an antenna suitable for wireless transceiving of electrical signals. Alternatively, the wire 7 may be configured to attach to device for transmission of electrical signals. Accordingly, the wire 7 may be dimensioned at various lengths, shapes, and configurations. In one embodiment, the connector 1 is made of translucent material with the exception of the valve 4, sensor 6, and wire 7.

The vent component 2 comprises an angled surface 19 extending from the vent body 11. The angled surface 19 comprises an opening 8 positioned adjacent to the sensor 6. A fin 12 extends along the angled surface 19 of the vent component 2 and over the opening 8 such that the fin 12 bisects the opening 8. The fin 12 includes a base 17 such that the base 17 and the fin 12 create a generally “T” shaped configuration, although other configurations are possible. The vent component 2 further comprises a vent interface 13 adjacent to the angled surface 19.

The trach component 3 comprises a trach interface 9 connected to a respiratory interface 10, wherein the valve 4 is positioned in between the trach interface 9 of the trach component 3 and the vent interface 13 of the vent component 2. The tube interface 5 of the vent component 2 and the respiratory interface 10 of the trach component 3 both are configured as substantially cylindrical and hollow structures.

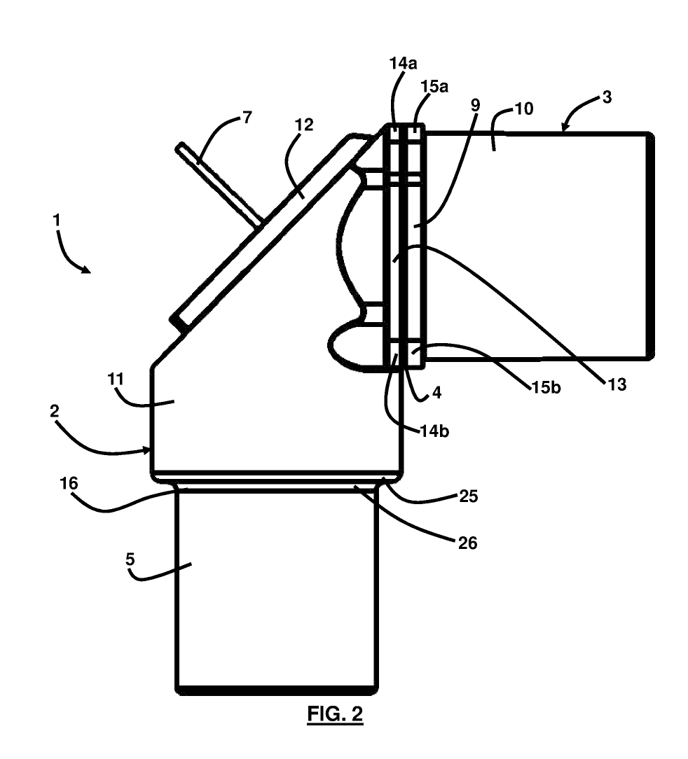

FIG. 2, with reference to FIG. 1, illustrates a front view of the connector 1 according to an embodiment herein. The neck 16 between the tube interface 5 and the vent body 11 may be configured as a cascading structure comprising a plurality of steps 25, 26 such that the outer diametrical width of the tube interface 5 is less than the outer diametrical width of the vent body 11. The vent interface 13 comprises a plurality of holes 14a-14d (only holes 14a and 14b are shown in FIG. 2), which are configured to accept pegs 15a-15d, respectively, of the trach interface 9 (only pegs 15a and 15b are shown in FIG. 2). The holes 14a-14d and pegs 15a-15d are configured to connect the vent component 2 to the trach component 3 with the valve 4 being held in pace therebetween.

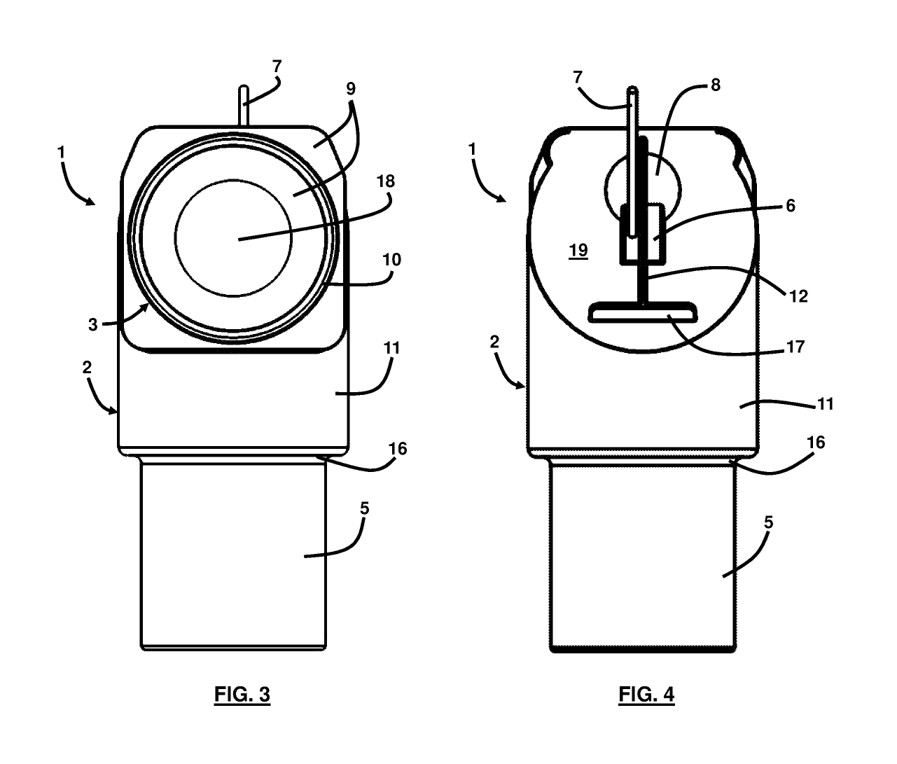

FIG. 3, with reference to FIGS. 1 and 2, illustrates a right side view of the connector 1 according to an embodiment herein. The respiratory interface 10 of the trach component 3 comprises a hollow channel 18, which extends through the entire length of the respiratory interface 10. The trach interface 9 is dimensioned as a substantially square shape configuration with the channel 18 positioned in the middle of the trach interface 9.

FIG. 4, with reference to FIGS. 1 through 3, illustrates a left side view of the connector 1 according to an embodiment herein. As shown, the sensor 6 is positioned adjacent to a substantially middle portion of the angled surface 19 of the vent component 2, such that a portion of the sensor 6 extends into the opening 8 of the vent component 2.

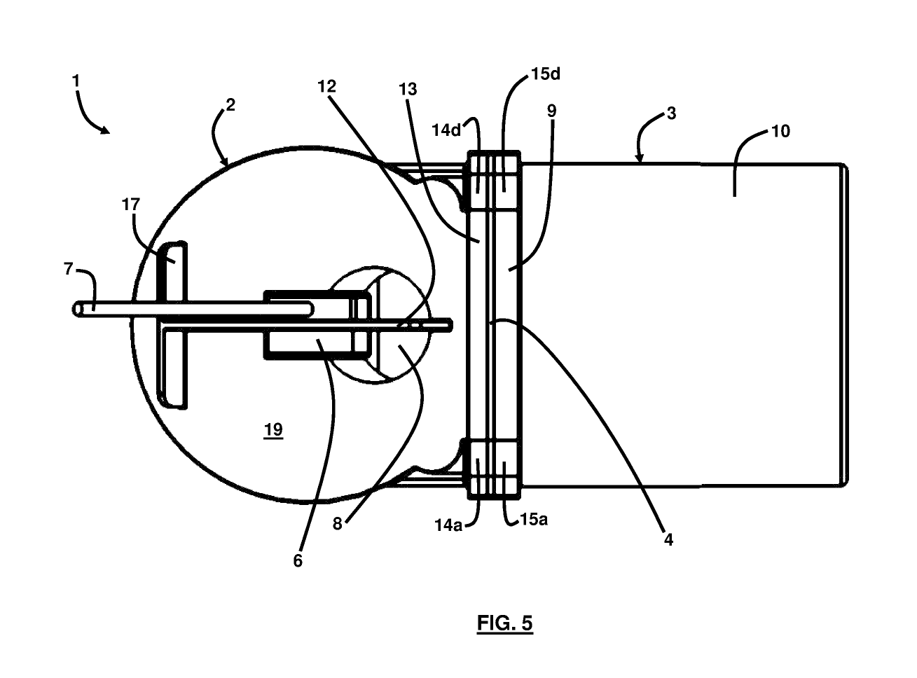

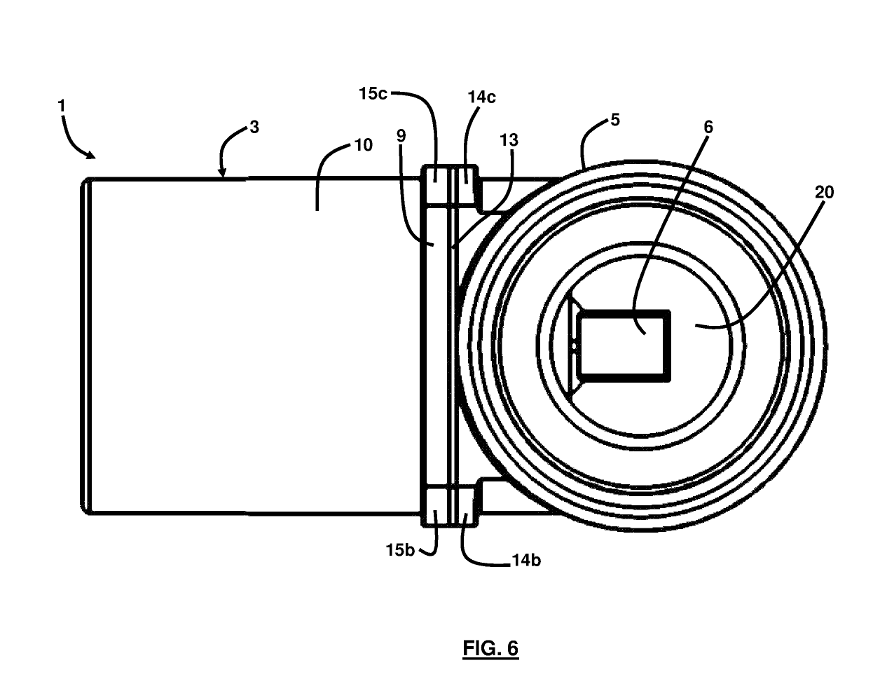

FIG. 5, with reference to FIGS. 1 through 4, illustrates a top view of the connector 1 according to an embodiment herein. The sensor 6 is configured to sense air flow coming from the channel 18 of the trach component 3 past the valve 4 and into the opening 8 of the vent component 2. FIG. 6, with reference to FIGS. 1 through 5, illustrates a bottom view of the connector 1 according to an embodiment herein. The tube interface 5 of the vent body 11 comprises a channel 20 through which air flows. The sensor 6 is in the path of the channel 20 in order to take air flow measurements.

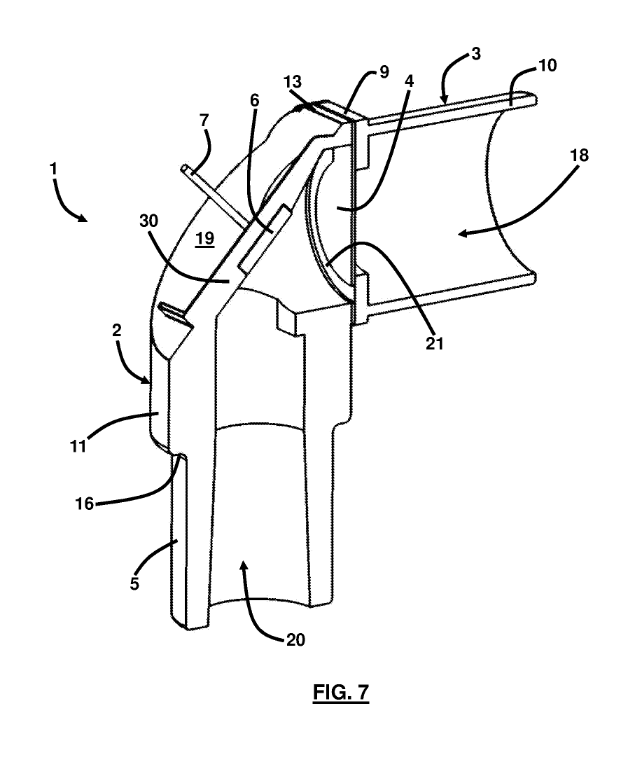

FIGS. 7 through 8, with reference to FIGS. 1 through 6, illustrate cross-sectional perspective views of the connector 1 according to an embodiment herein. The sensor 6 is seated in the angled portion 30 of the vent component 2. While the outer diametrical width of the tube interface 5 is less than the outer diametrical width of the vent body 11, the inner diametrical width of the tube interface 5 is substantially the same as the inner diametrical width of the vent body 11, in one embodiment. The valve 4 comprises a gap 21 configured in a substantially horseshoe shaped configuration, in one embodiment.

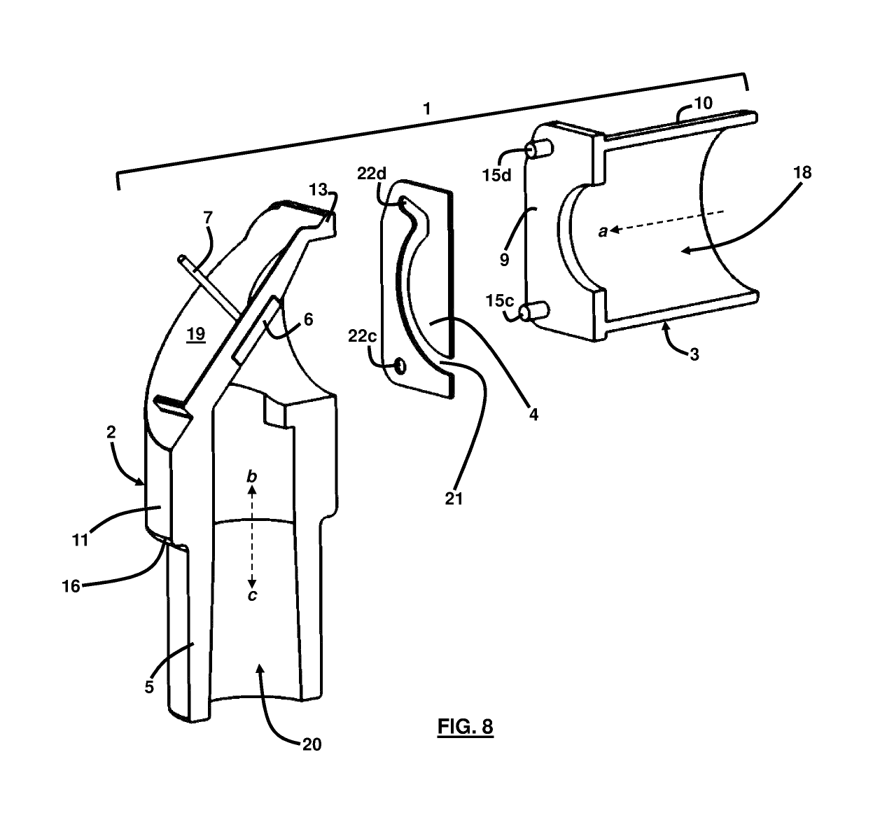

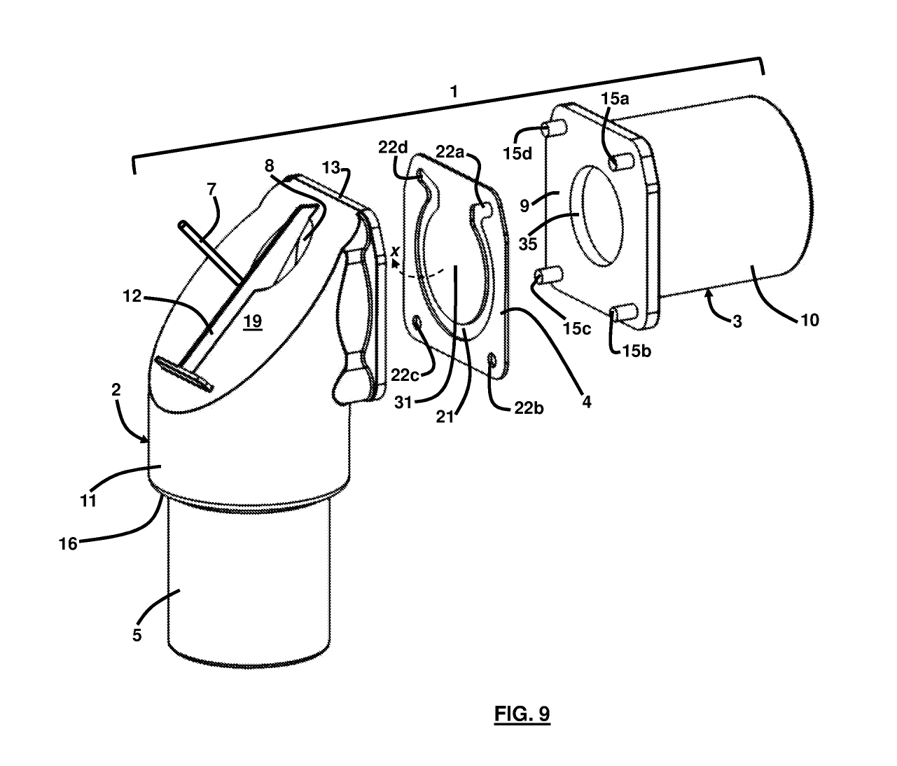

FIG. 9, with reference to FIGS. 1 through 8, illustrates an exploded perspective view of the connector 1 according to an embodiment herein. The valve 4 comprises a flexible rubber material such that the gap 21 permits the central portion 31 of the valve 4 to flap towards direction x, as air flows from the trach component 3. The diameter of the central portion 31 of the valve 4 is larger than the diameter of the hole 35 configured in the trach interface 9 of the trach component 3. The pegs 15a-15d of the trach component 3 are dimensioned and configured to fit through the slots/holes 22a-22d of the valve 4 to retain the valve 4 in place once the pegs 15a-15d are inserted into the holes 14a-14d, respectively, of the vent component 2 such that peg 15a is inserted through slot 22a and into hole 14a, peg 15b is inserted through hole 22b and into hole 14b, peg 15c is inserted through hole 22c and into hole 14c, and peg 15d is inserted through slot 22d and into hole 14d.

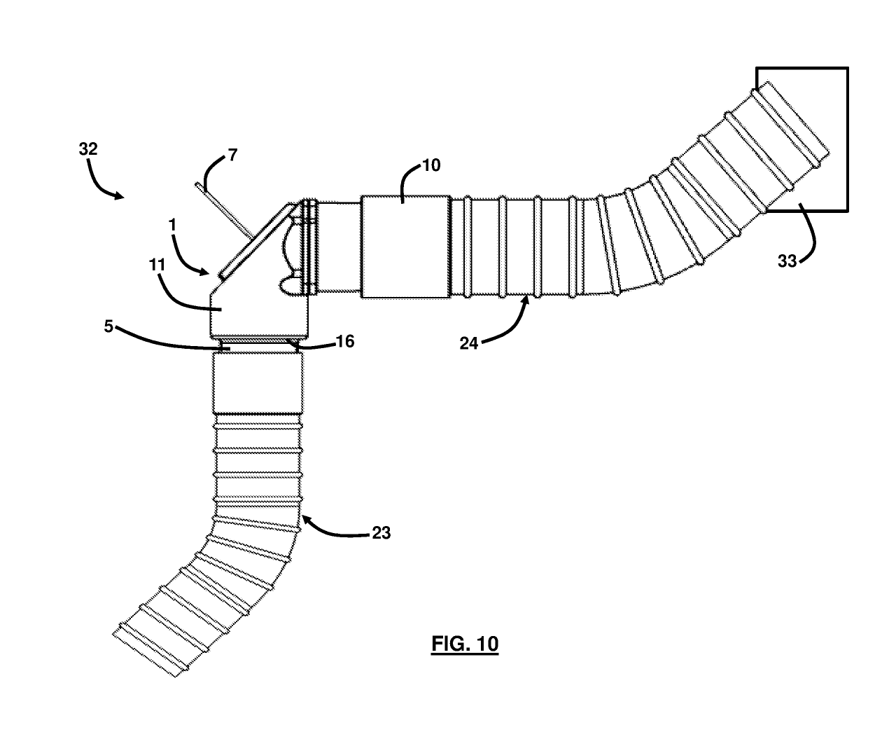

FIG. 10, with reference to FIGS. 1 through 9, illustrates a system 32 comprising the tracheostomy sensor connector 1 connected to a trach tube 23 and a respiratory device (e.g., BiPAP machine) tube 24 according to an embodiment herein. The trach tube 23 may be a conventional tube used for ventilating tracheostomy patients, and the respiratory device tube 24 may be a conventional tube connected to a respiratory device 33 (e.g., BiPAP machine). During inhalation, the respiratory device 33 (e.g., BiPAP machine) pushes air through tube 24 through the channel 18 of the respiratory interface 10 of the vent component 2. The central portion 31 of the valve 4 flaps open (in direction x shown in FIG. 9) as air is pushed through the vent body 11 in direction a (shown in FIG. 8) such that the central portion 31 of the valve 4 covers opening 8 of the vent body 11 to prevent air from escaping. Air is then pushed into the tube interface 5 and into the trach tube 23 and into the patient (not shown). Exhaled air from the patient enters the trach tube 23 into the tube interface 5, through the vent body 11 and out through the opening 8 as the central portion 31 of the valve 4 remains in the closed position during exhaling since air is not being pushed against it from the respiratory device 33 (e.g., BiPAP machine) at this time, and since the diameter of the central portion 31 of the valve 4 is larger than the diameter of the hole 35 configured in the trach interface 9 of the trach component 3, thus the central portion 31 of the valve 4 cannot move past the trach interface 9 as exhaled air is being pushed against it, and therefore all of the exhaled air is vented out of the opening 8. The sensor 6 detects the proper flow of air in the connector 1 both during an inhaling and exhaling process, and if the sensor 6 detects a lack or reduced amount of air flow in the connector 1 and/or reduced motion of the central portion 31 of the valve 4, then it generates a signal through the wire 7 to a remote device/machine (not shown) indicating a reduced level of air flow in the connector 1 and/or reduced level of motion of the valve 4. In one embodiment, the sensor 6 comprises a speaker 34 that outputs an audio signal indicating the reduced level of air flow in the connector 1 and/or reduced motion of the valve 4. In another embodiment, the sensor 6 comprises a light-emitting diode (LED) 36 that functions as a light indicator indicating whenever there is a reduced level of air flow in the connector 1 and/or reduced level of motion of the valve 4.

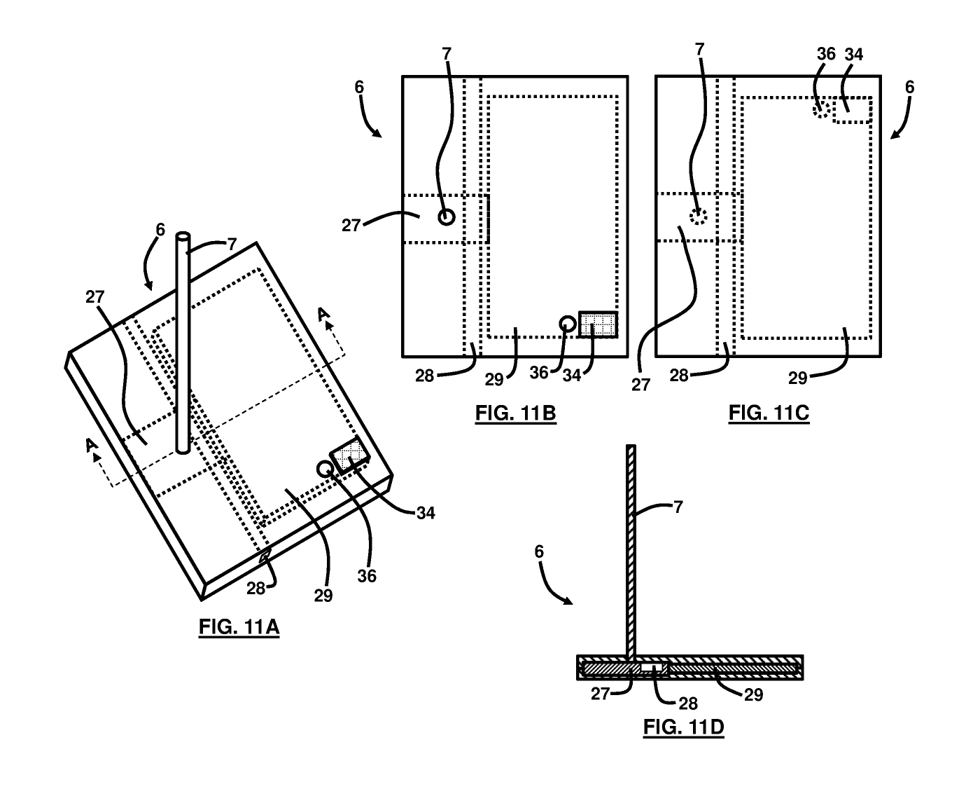

FIGS. 11A through 11D, with reference to FIGS. 1 through 10, illustrate various views of the sensor 6 according to an embodiment herein. The sensor 6 can measure the air flow or detect motion of the flapping central portion 31 of the valve 4 using an embedded flowmeter 28 and an air pressure/motion detector 29 to allow for air flow, differential pressure, and motion sensing. In an example embodiment, the sensor 6 may include a module 27 to provide power (e.g., battery) for the detector 29 and to electrically connect the wire 7 to the detector 29. If the wire 7 is configured as an antenna, then the signals from the detector 29 are wirelessly transmitted by the wire/antenna 7 to a receiving device (not shown) for processing.

Generally, the embodiments herein provide a respiratory device to tracheostomy tube connector 1 comprising a first (trach) component 3 comprising a first hollow channel 18 through which air is pumped from a respiratory device (e.g., BiPAP machine 33) in one direction a; and a second (vent) component 2 connected to the first component 3, wherein the second component 2 comprises a second hollow channel 20 through which air moves in two directions b, c to and from a tracheostomy tube 23; and an opening 8 through which air is vented upon return from the tracheostomy tube 23. The connector 1 further comprises a valve 4 positioned in between the first component 3 and the second component 2; and a sensor 6 seated in the second component 2 and adjacent to the opening 8, wherein the sensor 6 detects any of a flow of air in the second component 2 and a motion of the valve 4.

The sensor 6 may comprise an air flowmeter 28. The sensor 6 may comprise a detector 29. The detector 29 may detect air pressure in the second component 2. The detector 29 may detect the motion of the valve 4. The sensor 6 may comprise a wire 7 that transmits electrical signals to and from the detector 29. The sensor 6 may comprise a module 27. The module 27 may provide power to the sensor 6. The module 27 may electrically connect the wire 7 to the detector 29. The wire 7 may comprise and/or be embodied as an antenna, in one embodiment. The wire 7 may extend from the sensor 6 away from the second component 2. The sensor 6 may comprise a speaker 34 that outputs audio. The sensor 6 may comprise a light indicator 36.

The second component 2 may comprise a tube interface portion 5 configured to engage the tracheostomy tube 23; and a (vent) body portion 11 comprising the opening 8 and the sensor 6, wherein the second hollow channel 20 extends from the tube interface portion 5 to the body portion 11. An outer diametrical width of the tube interface portion 5 may be less than an outer diametrical width of the body portion 11. The connector 1 may further comprise a lip 16 separating the tube interface portion 5 from the body portion 11. The tracheostomy tube 23 does not connect to a respiration mask. The valve 4 may comprise a flexible central portion 31 that articulates from an open to closed position, wherein the flexible central portion 31 covers the opening 8 when the valve 4 is in the open position. When the valve 4 is in the closed position, air returning from the tracheostomy tube 23 may be prevented from entering into the first component 3 due to the flexible central portion 31 being in its original (e.g., non-open) position.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A respiratory device to tracheostomy tube connector comprising:
a first component comprising a first hollow channel through which air is pumped from a respiratory device in one direction;
a second component connected to said first component, wherein said second component comprises:
a second hollow channel through which air moves in two directions to and from a tracheostomy tube; and
an opening through which air is vented upon return from said tracheostomy tube;
a valve positioned in between said first component and said second component; and
a sensor seated in said second component and adjacent to said opening, wherein said sensor detects any of a flow of air in said second component and a motion of said valve.

2. The connector of claim 1, wherein said respiratory device comprises a Bilevel positive airway pressure (BiPAP) machine.

3. The connector of claim 1, wherein said sensor comprises an air flowmeter.

4. The connector of claim 1, wherein said sensor comprises a detector.

5. The connector of claim 5, wherein said detector detects air pressure in said second component.

6. The connector of claim 5, wherein said detector detects said motion of said valve.

7. The connector of claim 5, wherein said sensor comprises a wire that transmits electrical signals to and from said detector.

8. The connector of claim 8, wherein said sensor comprises a module.

9. The connector of claim 9, wherein said module provides power to said sensor.

10. The connector of claim 9, wherein said module electrically connects said wire to said detector.

11. The connector of claim 8, wherein said wire comprises an antenna.

12. The connector of claim 8, wherein said wire extends from said sensor away from said second component.

13. The connector of claim 1, wherein said sensor comprises a speaker that outputs audio.

14. The connector of claim 1, wherein said sensor comprises a light indicator.

15. The connector of claim 1, wherein said second component comprises:
a tube interface portion configured to engage said tracheostomy tube; and
a body portion comprising said opening and said sensor, wherein said second hollow channel extends from said tube interface portion to said body portion.

16. The connector of claim 15, wherein an outer diametrical width of said tube interface portion is less than an outer diametrical width of said body portion.

17. The connector of claim 15, further comprising a lip separating said tube interface portion from said body portion.

18. The connector of claim 1, wherein said tracheostomy tube does not connect to a respiration mask.

19. The connector of claim 1, wherein said valve comprises a flexible central portion that articulates from an open to closed position, and wherein said flexible central portion covers said opening when said valve is in said open position.

20. The connector of claim 19, wherein when said valve is in said closed position, air returning from said tracheostomy tube is prevented from entering into said first component.
